(19) United States
(12) Patent Application Publication
(10) Pub. No.: US 2005/0002884 A1
(43) Pub. Date: Jan. 6, 2005

(54) Hair treatment compositions

(76) Inventor: Marsella Jefferson, Mt. Pleasant, SC (US)

Correspondence Address: KATHLEEN M HARLESTON;THE HARLESTON LAW FIRM, 909 TALL PINE ROAD, MT PLEASANT, SC, 29464, US

(21) Appl. No.: 10/612,517

(22) Filed: Jul. 2, 2003

(51) Int. Cl. A61K 7/06; A61K 7/075; A61K 7/08; A61K 35/78

(52) U.S. Cl. 424/70.1; 424/70.23; 424/74; 424/757; 424/766; 424/745; 424/735

(57) ABSTRACT

A hair treatment composition includes: (a) from about 5 to about 30 weight % of at least one aromatic essential oil; (b) from about 20 to about 80 weight % of petroleum jelly; (c) from about 2 to about 20 weight % of lecithin; and (d) from about 5 to about 40 weight % of water; wherein the ratio of aromatic essential oil and water: petroleum jelly is between about 1:10 and about 1:1. Also included is a process of making a stable, viscous hair treatment composition, including: (a) boiling water containing at least one aromatic essential oil; (b) allowing the aromatic oil/water mixture to cool; (c) beating together petroleum jelly, and the essential oil/water mixture in a ratio of between about 1:10 and about 1:1; (d) beating lecithin into the mixture; and (e) beating at least one aromatic essential oil into the mixture.

### CROSS REFERENCE TO RELATED DOCUMENT

This invention was described in Disclosure Document Number 504153, which was mailed to the U.S. Patent & Trademark Office on Oct. 23, 2001.

### BACKGROUND OF THE INVENTION

1. Technical Field

The present invention is a viscous hair treatment composition, more particularly a leave-in hair conditioning composition, and a process for making it.

2. Background Information

The increased use of late of hair extensions and abrasive hair coloring chemicals and hair products have greatly damaged the hair of the ladies or men who use them as beauty aids. It is ironic that the very thing that improves one's appearance in the short run can cause damage, which is sometimes irreparable, in the long run. There is a need in the hair care industry for a hair treatment composition, particularly for African-Americans, that will ameliorate this damage and leave hair with a soft feel and a lustrous appearance.

The hair treatment compositions of the present invention have been found to accomplish the following after repeated use:

1) treat and repair damaged hair caused by overexposure to sun, other environmental factors, and/or hair coloring chemicals;

2) effectively reduce or prevent scalp itching;

3) provide moisture, smoothness, and softness to the hair, as well as good wet or dry combability;

4) provide an excellent conditioning effect for a long-lasting luster; and/or

5) thicken and improve the texture of the hair.

Without meaning to be bound by theory, the present compositions are also believed to stimulate hair follicular growth and hair retention, thereby combating baldness.

### BRIEF SUMMARY OF THE INVENTION

The present invention encompasses a viscous hair treatment composition comprising:

(a) from about 5 to about 30 weight % of at least one aromatic essential oil;

(b) from about 20 to about 80 weight % of petroleum jelly;

(c) from about 2 to about 20 weight % of lecithin; and

(d) from about 5 to about 40 weight % of water;

wherein the ratio of aromatic essential oil and water: petroleum jelly is between about 1:10 and about 1:1. Vinegar is a preferred additional ingredient herein.

The present hair treatment compositions are made of natural ingredients. With dedicated usage, they are believed to repair damaged hair, stimulate the scalp (possibly to increase hair growth), maintain healthy hair, and leave a pleasant aroma.

Also included herein is a process of making a stable viscous hair treatment composition, comprising:

(a) boiling a mixture of from about 2 to about 20 weight % of at least one aromatic essential oil, and from about 80 to about 98 weight % of water;

(b) allowing the aromatic oil/water mixture to cool to room temperature;

(c) beating together from about 20 to about 80 weight % of petroleum jelly, and from about 5 to about 40 weight % of the essential oil/water mixture in a ratio of between about 1:10 and about about 1:1;

(d) beating into the mixture from about 2 to about 20 weight % of lecithin; and

(e) beating into the mixture from about 2 to about 20 weight % of at least one aromatic essential oil.

### DESCRIPTION OF THE INVENTION

Hair treatment compositions herein include from about 5 to about 30, more preferably from about 10 to about 20, weight %, of a conditioning aromatic essential oil, particularly almond oil, basil oil, clary sage oil, grape seed oil, jojoba oil, lavender oil, oregano oil, peppermint oil, rosemary oil, thyme oil, and/or teatree oil. By “aromatic” is meant that the oil emits a definite and pleasing aroma. The most preferred essential oils for use in the hair treatment compositions herein are almond oil, grape seed oil, jojoba oil, lavender oil, rosemary oil, and peppermint oil. Almond oil is not required for an effective formula herein, though.

Regarding specific essential oils, lavender oil is a potent fragrance used in aromatherapy. It is distilled from the lavender plant, Lavendula officionalis. Thyme oil is also commonly used in aromatherapy. It is made from red thyme, Thymus vulgaris. Basil oil is a sweet tasting oil commonly used in flavorings and in aromatherapy. It is distilled from basil leaves, Ocunium basillicum. Clary (“clear eye”) sage oil is also commonly used in aromatherapy. It is distilled from clary sage, Salvia sclarea. Peppermint oil made from the peppermint plant Mentha piperita was used by the ancient Greeks and Romans. Rosemary oil is commonly cold pressed from the rosemary plant, Rosemarinus officinalis. It has been used as a flavoring or fragrance in aromatherapy. Aside from their obvious aroma benefits and conditioning effect on the hair and scalp, the distilled essential oils used herein are believed to include enzymes and amino acids that benefit hair.

A second component for use in the compositions herein is petroleum jelly. The present formulas include from about 20 to about 80, more preferably from about 50 to about 70, weight % of petroleum jelly, or petrolatum jelly.

A third component for use in the compositions herein is lecithin, or another suitable emulsifier. The present formulas include from about 2 to about 20, more preferably from about 5 to about 15, weight % of lecithin.

A preferred additional ingredient for inclusion herein is virgin olive oil. The present formulas include from about 0.5 to about 10, more preferably from about 1 to about 5, weight % of virgin olive oil.

A preferred additional ingredient for inclusion herein is vinegar, preferably apple cider vinegar. The present formulas preferably include from about 2 to about 20, more preferably from about 5 to about 15, weight % of vinegar. Vinegar is believed to act as a preservative in the composition, helps the ingredients of the composition mix more evenly, and has a cleansing action on the hair.

Optional additional components for inclusion in the hair treatment compositions herein include: aloe vera gel, which is made from the aloe vera plant; citric acid; and natural or synthetic beeswax. From about 2 to about 30 weight % of aloe vera gel or beeswax may be included in the compositions of the present invention. From about 0.5 to about 5 weight % of citric acid is preferably included in the present composition as a preservative.

Another component for optional inclusion in the present composition is peppermint herbal infusion. The peppermint herbal infusion is preferably made by placing several stalks of peppermint into a tea-type strainer and placing the strainer in a quart of water. The water is brought to a boil and then cooled. A small amount of peppermint herbal infusion in the composition provides a tingling, stimulating feeling and an additional refreshing aroma. This ingredient is believed to strengthen and open pores in the scalp.

No ethoxylated fatty acids, mineral oils, or silicone are required herein. No surfactants are required herein.

Also included herein is a process of making a stable viscous hair treatment composition, comprising:

(a) boiling a mixture of from about 2 to about 20 weight % of at least one aromatic essential oil, and from about 80 to about 98 weight % of water;

(b) allowing the aromatic oil/water mixture to cool to room temperature;

(c) beating together from about 20 to about 80 weight % of petroleum jelly, and from about 5 to about 40 weight % of the essential oil/water mixture in a ratio of between about 1:10 and about 1:1;

(d) beating into the mixture from about 2 to about 20 weight % of lecithin; and

(e) beating into the mixture from about 2 to about 20 weight % of at least one aromatic essential oil.

These ingredients are evenly distributed in the present hair treatment composition. No heating is required for even distribution of the ingredients.

The process preferably further includes the steps of:

(f) beating into the mixture from about 2 to about 20 weight % of apple cider vinegar; and/or

(g) beating into the mixture from about 2 to about 20 weight % of at least one additional aromatic essential oil (see above).

To make the hair treatment composition, several of the essential oils (see above), preferably almond oil and rosemary oil, are dropped into several gallons of water in a heating vessel. The mixture is heated to a boil, then allowed to cool to room temperature and stirred. This essential oil/water infusion is beaten into the petroleum jelly for between about two and fifteen minutes, preferably using an electric beater at a medium setting, in a ratio of between about 1:10 and about 1:1, more preferably between about 1:1 and about 1:5, most preferably about 1:2. The hair treatment composition of the present invention is preferably stored in a cool location so that it remains stable in its gel-like state. The lecithin and vinegar, most preferably apple cider vinegar, are also beaten into the whipped mixture for several additional minutes. The essential oils (preferably five to ten of them) are then beaten into the whipped mixture using the same beater and setting in any order for between about five and fifteen minutes.

The present composition is a viscous, homogeneous mixture with a creamy appearance like an emulsion. It has the approximate consistency and color of vanilla pudding. No additional perfumes, colorants, or opacifiers are needed in the formula. Normally, a dime-sized amount of the present composition is rubbed between the palms or fingertips, then distributed through the hair and massaged on the scalp. The composition is easily applied and feels soothing on the scalp.

Daily use of the present hair treatment composition for a week or more has been found to: (1) improve the appearance of previously damaged hair; (2) reduce dry scalp and scalp itching; (3) moisturize, smooth, and soften the user's hair; (4) provide good combability; (5) provide long-lasting luster; and/or (6) improve hair texture. The present composition can be used on all hair types, but is particularly effective on curly, thick hair.

Without meaning to be bound by theory, it is believed that the hair follicles and papilla are supplied with nerves and blood vessels, and the growth of hair occurs at the point where the follicle and the papilla meet, and that the treatment compositions herein stimulate the growth of hair follicles, thereby helping to combat impending baldness.

The following examples are intended to further illustrate the invention and facilitate its understanding. These examples are given solely for the purposes of illustration and are not to be construed as limiting the present invention in any way.

### EXAMPLE I

A hair treatment composition is formulated by mixing the following ingredients in a mixing vessel:

| Ingredient | Amount |
|---|---|
| Petroleum jelly | 3150 ml. |
| Essential oil/water infusion | 1440 ml. |
| Apple cider vinegar | 480 ml. |
| Almond Oil | 480 ml. |
| Lecithin | 480 ml. |
| Grape seed oil | 240 ml. |
| Virgin olive oil | 60 ml. |
| Jojoba oil | 15 ml. |
| Rosemary oil | 5 ml. |
| Lavender oil | 10 ml. |
| Peppermint oil | 5 ml. |
| Basil oil | 2.5 ml. |
| Clary sage oil | 2.5 ml. |
| Teatree oil | 7.5 ml. |

### Process

To make the hair treatment composition, 70 milliliters of almond oil and 5 milliliters of rosemary oil are dropped into two gallons of water in a heating vessel. The mixture is heated to a boil, then allowed to cool to room temperature and stirred. 1440 milliliters of this essential oil/water infusion is whipped into 3150 milliliters of petroleum jelly for about two minutes using an electric beater on a medium setting, making a viscous mixture having the approximate consistency and color of vanilla pudding. 480 milliliters of lecithin and 480 milliliters of apple cider vinegar are also beaten into the whipped mixture for several additional minutes. The ten oils are then beaten into the whipped mixture in any order for another two minutes or so.

A viscous, stable, whipped composition is formed. It is recommended that the composition be kept in a cool location so that it is not exposed to a temperature greater than about 75 or 80 degrees Fahrenheit.

### Use

A dime-sized amount of the whipped composition is worked into the hair and scalp each night for a recommended period, usually one week. After this time, the user's hair will have a long-lasting luster and soft feel and be easy to comb through and manage.

### EXAMPLE II

A hair treatment composition is formulated by beating the following ingredient into the composition of Example I:

| Ingredient | Amount |
|---|---|
| Peppermint Herbal Infusion | 15 ml. |

### Process

The peppermint herbal infusion is made by placing several stalks of peppermint into a tea-type strainer and placing the strainer in a quart of water. The water is brought to a boil and then cooled. 15 milliliters of the peppermint herbal infusion is beaten into the composition of Example I along with the oils.

### Use

A dime-sized amount of the whipped composition is worked into the hair and scalp each night for a recommended period, usually one week. The peppermint herbal infusion adds an additional pleasant smell to the composition and to the user's hair after use of the composition. It may cause a slight, pleasant tingling sensation on the scalp when it is applied.

### EXAMPLE III

A leave-in conditioner for use after shampooing hair is formulated by beating the following ingredient into the composition of Example I:

| Ingredient | Amount |
|---|---|
| Aloe Vera Gel | 240 ml. |

Tom make this formula, 240 milliliters of aloe vera gel is beaten into the composition of Example I along with the essential oils.

A small amount of this leave-in conditioner is applied to the hair just after shampooing the hair for a smooth feel and long-lasting luster and shine.

### EXAMPLE IV

A hair treatment composition is formulated by mixing the following ingredients in a mixing vessel:

| Ingredient | Amount |
|---|---|
| Petroleum jelly | 3000 ml. |
| Essential oil/water infusion | 1000 ml. |
| Lecithin | 480 ml. |
| Grape seed oil | 240 ml. |
| Jojoba oil | 10 ml. |
| Rosemary oil | 5 ml. |
| Lavender oil | 10 ml. |
| Peppermint oil | 5 ml. |
| Teatree oil | 10 ml. |
| Citric acid | 50 ml. |

### Process

To make the hair treatment composition, 50 milliliters of jojoba oil and 5 milliliters of rosemary oil are dropped into two gallons of water in a heating vessel. The mixture is heated to a boil, then allowed to cool to room temperature and stirred. 1000 milliliters of this essential oil/water infusion is whipped into 3000 milliliters of petroleum jelly for about two minutes using an electric beater at medium speed. 480 milliliters of lecithin are also beaten into the whipped mixture for about two additional minutes. The essential oils are then beaten into the whipped mixture in any order for another two minutes or so. The citric acid is beaten into the composition last.

A whipped, creamy composition is formed. It is recommended that the composition be kept in a cool location so that it is not exposed to a temperature greater than about 75 or 80 degrees Fahrenheit.

### Use

A dime-sized amount of the whipped composition is worked into the hair and scalp each night for a recommended period, usually one week. After this time, the user's hair will have a long-lasting luster and soft feel and be easy to comb through and manage.

### EXAMPLE V

A hair treatment composition is formulated by mixing the following ingredients in a mixing vessel:

| Ingredient | Amount |
|---|---|
| Petroleum jelly | 3000 ml. |
| Apple cider vinegar | 480 ml. |
| Lecithin | 480 ml. |
| Grape seed oil | 240 ml. |
| Jojoba oil | 20 ml. |
| Rosemary oil | 5 ml. |
| Lavender oil | 10 ml. |
| Teatree oil | 10 ml. |
| Purified water | 1000 ml. |

### Process

To make the hair treatment composition, 1000 milliliters of purified water is whipped into 3000 milliliters of petroleum jelly for several minutes using a conventional electric beater on a medium setting. 480 milliliters of lecithin and 480 milliliters of apple cider vinegar are also beaten into the whipped mixture for several additional minutes. The essential oils are then beaten into the whipped mixture in any order for another two minutes or so.

A thick, creamy, homogeneous composition is formed. It is recommended that the composition be kept in a cool location so that it is not exposed to a temperature greater than about 75 or 80 degrees Fahrenheit.

### Use

About five milliliters of the whipped composition is worked into the hair and scalp each night for a recommended period, usually one week. After this time, the user's hair will have a long-lasting luster and soft feel and be easy to comb through and manage.

While preferred embodiments of the invention have been described using specific terms, this description is for illustrative purposes only. It will be apparent to those of ordinary skill in the art that various modifications, substitutions, omissions, and changes may be made without departing from the spirit or scope of the invention, and that such are intended to be within the scope of the present invention as defined by the following claims. It is to be understood that any amounts given herein are illustrative, and are not meant to be limiting. It is intended that the doctrine of equivalents be relied upon to determine the fair scope of these claims in connection with any other person's product which fall outside the literal wording of these claims, but which in reality do not materially depart from this invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

1. A composition for the treatment of hair, comprising:
(a) from about 5 to about 30 weight % of at least one aromatic essential oil;
(b) from about 20 to about 80 weight % of petroleum jelly;
(c) from about 2 to about 20 weight % of lecithin; and
(d) from about 5 to about 40 weight % of water;
wherein the ratio of aromatic essential oil and water:petroleum jelly is between about 1:10 and about 1:1.

2. A hair treatment composition according to claim 1, wherein the essential oil is jojoba oil.

3. A hair treatment composition according to claim 1, wherein the ratio of the petroleum jelly to the essential oil mixture is about 1:2 to about 1:4.

4. A hair treatment composition according to claim 3, wherein the essential oil is grape seed oil.

5. A hair treatment composition according to claim 3, wherein the essential oil is teatree oil.

6. A hair treatment composition according to claim 3, wherein the essential oil is almond oil.

7. A hair treatment composition according to claim 3, further comprising from about 0.02 to about 0.5 milliliters of basil oil.

8. A hair treatment composition according to claim 3, further comprising from about 0.02 to about 1 milliliter of clary sage oil.

9. A hair treatment composition according to claim 3, further comprising from about 0.02 to about 1 milliliter of lavender oil.

10. A hair treatment composition according to claim 1, further comprising from about 0.02 to about 1 milliliter of peppermint oil or peppermint herbal infusion.

11. A hair treatment composition according to claim 2, further comprising from about 0.02 to about 1 milliliter of rosemary oil.

12. A hair treatment composition according to claim 11, further comprising from about 0.02 to about 1 milliliter of thyme oil.

13. A hair treatment composition according to claim 3, further comprising from about 2 to about 20 weight % of apple cider vinegar.

14. A hair treatment composition according to claim 3, further comprising from about 0.5 to about 5 weight % of citric acid.

15. A hair treatment composition according to claim 14, further comprising from about 0.5 to about 10 weight % of olive oil.

16. A process of making a viscous hair treatment composition, comprising:
(a) boiling a mixture of from about 2 to about 20 weight % of at least one aromatic essential oil, and from about 80 to about 98 weight % of water;
(b) allowing the aromatic oil/water mixture to cool to room temperature;
(c) beating together from about 20 to about 80 weight % of petroleum jelly, and from about 5 to about 40 weight % of the essential oil/water mixture in a ratio of between about 1:10 and about 1:1;
(d) beating into the mixture from about 2 to about 20 weight % of lecithin; and
(e) beating into the mixture from about 2 to about 20 weight % of at least one aromatic essential oil.

17. A process according to claim 16, wherein the ratio of the petroleum jelly to the essential oil mixture is between about 1:1 and about 1:5.

18. A process according to claim 16, further comprising the step of:
(f) beating into the whipped mixture from about 2 to about 20 weight % of apple cider vinegar.

19. A process according to claim 18, further comprising the step of:
(g) beating into the whipped mixture from about 2 to about 20 weight % of at least one additional aromatic essential oil.

20. A process according to claim 18, wherein the essential oil is jojoba oil.
